(19) United States
(12) Patent Application Publication
(10) Pub. No.: US 2022/0379060 A1
(43) Pub. Date: Dec. 1, 2022

(54) PATIENT INTERFACE WITH A SEALING DEVICE COMPRISING A SUPPORT MEMBRANE AND A SEALING MEMBRANE

(71) Applicant: Loewenstein Medical Technology S.A., Luxembourg (LU)

(72) Inventors: Martin EIFLER, Glueckstadt (DE); Anja Gottschewski, Hamburg (DE)

(21) Appl. No.: 17/664,695

(22) Filed: May 24, 2022

(30) Foreign Application Priority Data
May 28, 2021 (DE) 102021002769.8

(51) Int. Cl. A61M 16/06

(52) U.S. Cl. CPC A61M 16/0605

(57) ABSTRACT

A patient interface with a sealing device, the sealing device comprising a support membrane and a sealing membrane. The sealing membrane is designed at least in part to bear on the skin of a patient and the support membrane extends at least in part adjacent to the sealing membrane. The distance between the support membrane and the sealing membrane is at least in part such that an interspace is formed.

### CROSS-REFERENCE TO RELATED APPLICATIONS

The present application claims priority under 35 U.S.C. § 119 of German Patent Application No. 102021002769.8, filed May 28, 2021, the entire disclosure of which is expressly incorporated by reference herein.

### FIELD OF THE INVENTION

The present invention relates to a patient interface with a sealing device comprising a support membrane and a sealing membrane.

### SUMMARY OF THE INVENTION

The invention provides a patient interface having a sealing device. The sealing device comprises a support membrane and a sealing membrane, the sealing membrane being designed at least in part to bear on the skin of a patient. The support membrane extends at least in part adjacent to the sealing membrane, and the distance between the support membrane and the sealing membrane is at least in part configured in such a way that an interspace is formed.

Preferably, the distance between the support membrane and the sealing membrane is, at least in part, from about 1 mm to about 5 mm.

The patient interface may also be characterized in that the sealing device has an edge profile with a base, the sealing membrane and the support membrane extending, spaced apart from each other, starting from the common base.

The patient interface may also be characterized in that the sealing device has an approximately U-shaped edge profile, which forms a fastening region for connecting the sealing device by form-fit engagement to a mask body.

The patient interface may also be characterized in that the fastening region is formed by a peripheral open groove.

The patient interface may also be characterized in that the edge profile has two profile walls which form the groove.

The patient interface may also be characterized in that the groove has a funnel shape.

The patient interface may also be characterized in that the groove has a funnel shape by virtue of the fact that at least one of the profile walls has a beveled wall portion which, with its bevel (beveled wall portion), is set back from the opposite profile wall.

The patient interface may also be characterized in that the profile wall has a lug.

The patient interface may also be characterized in that the profile wall has a beveled wall portion which faces the base of the U-shaped edge profile.

The patient interface may also be characterized in that the beveled wall portion has an undercut for the widening on the counter-profile of the mask body, and the edge profile can thus receive a counter-profile of a mask body.

The patient interface may also be characterized in that the membranes start from the edge profile and point in a direction away from the groove.

The patient interface may also be characterized in that the support membrane is shorter than the sealing membrane, and that the support membrane is at least in part covered by the sealing membrane.

It is thereby ensured that the patient's face is usually only in contact with the sealing membrane.

The patient interface may also be characterized in that a sealing membrane originates from the edge profile all about the periphery.

The patient interface may also be characterized in that in some regions there is no support membrane originating from the edge profile. Therefore, the support membrane is not continuous.

The nose bridge region, i.e., the region of the sealing device designed to bear on the bridge of the nose, may be cut out from the support membrane.

The chin region, i.e., the region of the sealing device designed to bear on the chin or the upper lip or the lower lip, may be cut out from the support membrane.

The patient interface may also be characterized in that a contact region to a patient's skin is formed by the sealing membrane, which may be formed as a sealing lip in the contact region.

The patient interface may also be characterized in that the sealing membrane extends in a radiating fashion, starting from the base of the edge profile, and then bends round in one direction (to an inner space into which the nose or mouth is partially inserted), the sealing membrane at least in part forming a right angle to itself.

The patient interface may also be characterized in that the support membrane is at least in part of thicker cross section than the sealing membrane.

The patient interface may also be characterized in that the membranes, spaced apart from each other, originate in the base, the sealing membrane being designed as a continuation of the wall, the wall thickness of the sealing membrane being smaller than the wall thickness of the wall.

The patient interface may also be characterized in that the wall thickness of the sealing membrane, in its further course, has a constant or only slightly decreasing wall thickness.

The patient interface may also be characterized in that the support membrane, substantially with an offset, is designed as a continuation of the wall.

The patient interface may also be characterized in that the wall thickness of the support membrane substantially corresponds to the wall thickness of the wall, and the wall thickness of the support membrane, in its further course (toward the sealing lip), has a decreasing wall thickness.

The patient interface may also be characterized in that the membranes form an elastic jacket-shaped wall portion and end in sealing lips.

The patient interface may also be characterized in that the wall thickness of the sealing membrane, in the course toward the sealing lip, has a constant or only slightly decreasing wall thickness, the wall thickness of the sealing membrane being in the range of from about 0.15 mm to about 0.6 mm.

Preferably, the wall thickness is in the range of from about 0.25 mm to about 0.45 mm, particularly preferably in the range from about 0.3 mm to about 0.4 mm.

The patient interface may also be characterized in that the wall thickness of the support membrane is in the range from about 2.9 mm to about 1.4 mm at the location where the support membrane originates from the base.

Preferably, the wall thickness is in the range from about 2.1 to about 1.6 mm, particularly preferably in the range from about 2.0 mm to about 1.7 mm, very particularly preferably about 1.8 mm.

The patient interface may also be characterized in that the wall thickness of the support membrane in the region of the sealing lip is in the range from about 0.7 mm to about 0.25 mm.

Preferably, the wall thickness is in the range from about 0.65 mm to about 0.4 mm, particularly preferably in the range from about 0.6 mm to about 0.45 mm, very particularly preferably about 0.5 mm.

The patient interface may also be characterized in that the wall thickness of the sealing lips is substantially identical.

The patient interface may also be characterized in that the distance between the sealing membrane and the support membrane at the base is from about 0.5 mm to about 3.5 mm, and from there the membranes extend apart from each other with an angle of greater than about 1°, preferably of from about 1.3° to about 4°, particularly preferably of about 3°.

The slight angle has the effect that the support membrane and the sealing membrane do not adhere to each other or run together but instead diverge. In this way, respiratory gas is able to flow through the interspace at least partially surrounded by the membranes. The flow of respiratory gas through the interspace at least partially surrounded by the membranes has at least two advantageous effects. Firstly, the therapeutic pressure applied can blow the sealing membrane at least slightly outward or press it against the patient's face. Secondly, an accumulation of condensing moisture from the respiratory air or the respiratory gas is at least lessened or partially prevented by the flow through the interspace.

The patient interface may also be characterized in that, over the region of the common extent, the distance between the membranes is substantially from about 1 mm to about 4 mm, preferably from about 1.4 mm to about 3 mm, particularly preferably from about 1.5 mm to about 1.8 mm or from about 1.5 mm to about 2 mm.

The patient interface may also be characterized in that the sealing device is produced from silicone or another elastic material, the sealing device comprises a support membrane and a sealing membrane, the sealing membrane forms an elastic jacket-like wall portion which delimits the sealing device to the outside and at least partially covers or overlaps the support membrane, and the elasticity of the elastic jacket-like wall portion permits a movement of the membranes relative to the fastening region of the sealing device formed by the edge profile, in particular a displacement perpendicular to the longitudinal axis and/or a tilting or pivoting about axes perpendicular to the longitudinal axis.

The patient interface may also be characterized in that the mask body and the sealing device together form an inner space or respiratory gas space into which the face of the user/patient at least partially protrudes during use of the mask on the face, the inner space of the patient interface is connected to the outer region of the patient interface in a gas-conducting manner via an exhalation system, and, by way of a hose connector, the patient interface is connected to a ventilator in a gas-conducting manner via a breathing hose, the sealing device having a contact region which is designed to bear sealingly on a patient's face and is formed principally by the sealing lip.

The patient interface may also be characterized in that the contact region has a profile which is designed such that the contact region is designed to bear on a nose bridge and to bear along a nasolabial fold and along an upper lip or lower lip or a chin region of a patient.

The support membrane may usually not be apparent, or be at least less apparent, in the nose bridge region or in the region of the upper lip or lower lip or in the chin region.

### BRIEF DESCRIPTION OF THE DRAWINGS

In the accompanying drawings:

FIG. 1 shows an illustrative embodiment of the patient interface as a mask with an exhalation system;

FIG. 2 shows a schematic representation of a sealing device with different sectional planes;

FIG. 3 shows a sealing device in a plan view from above;

FIG. 4 shows a sealing device in a plan view from below;

FIG. 5 shows another sealing device in a plan view from above;

FIG. 6 shows another sealing device in a plan view from below;

FIG. 7 shows a section through a sealing device according to FIG. 3 in a nose bridge region;

FIG. 8 shows a section through a sealing device according to FIG. 3 at the lower end of the nasolabial region;

FIG. 9 shows sealing membranes which are at least partially curved or rounded;

FIG. 10 shows two sealing membranes which originate in a base and, spaced apart from each other, point downward and end in two sealing lips;

FIG. 11 shows a sealing device which has an approximately U-shaped edge profile which forms a fastening region for connecting the sealing device to a mask body;

FIG. 12 shows another sealing device with a U-shaped edge profile and form-fit connection of the sealing device to a mask body;

FIG. 13 shows a section through a sealing device according to FIG. 3, which section thus runs through the vertical of the sealing device in the y direction; and

FIG. 14 shows a further illustrative embodiment of a sealing device in a section along a plane which runs transverse to the longitudinal axis of the sealing device.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

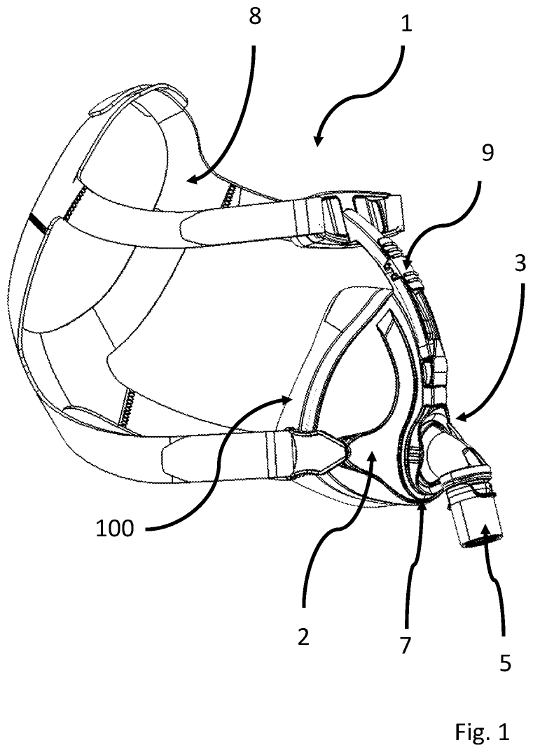

FIG. 1 shows an illustrative embodiment of the patient interface 1 as a mask with an illustrative embodiment of the exhalation system 3. The patient interface 1 comprises, for example, a mask body 2 on which a forehead rest 9 and a head harness 8 are arranged. The closure piece 7, for example together with parts of the mask body 2, forms the exhalation system 3. For example, the closure piece 7 is also configured such that a joint head of a ball-and-socket joint 6 can be received. A hose connector 5 is connected to the patient interface 1 via the ball-and-socket joint 6. A sealing device 100, designed to bear with one surface on the face of a user/patient, is connected to the mask body 2. Moreover, the patient interface comprises a sealing device 100 designed to receive at least parts of the face, for example the nose and/or mouth. The mask body 2 and the sealing device 100 together enclose an inner space (hereinafter simply the inner space of the patient interface 1) into which the face of the user/patient at least partially protrudes during use of the mask on the face. The sealing device 100 is connected to the mask body 2 for example via a profile 200. In some embodiments, the sealing device 100 is also part of the mask body. The inner space of the patient interface 1 is connected to the outer region of the patient interface 1 in a gas-conducting manner via the exhalation system 3. By way of the hose connector 5, the patient interface 1 can be connected to a ventilator in a gas-conducting manner, for example via a breathing hose. Thus, by way of the hose connector 5, respiratory gas can for example be delivered to and/or from the inner space of the patient interface 1 and the user/patient. For example, the patient interface 1 is designed as a full-face mask, such that at least the nose and mouth of the user are enclosed by the sealing device 100. However, the sealing device 100 according to the invention can also be used in other patient interfaces 1, for example nose masks, tracheal cannulas/tracheal tubes and/or nasal cannulas.

For example, respiratory gas is conveyed from a ventilator into the inner space 400 of the patient interface 1 through the hose connector 5 and the ball-and-socket joint 6 in order to assist the breathing of the user/patient. The conveying of the respiratory gas is controlled, for example, via the pressure or the flow. Respiratory gas can deliberately escape continuously through the optional exhalation system 3, as a result of which exhaled respiratory gas from the patient/user is also conveyed or washed out of the inner space of the patient interface 1. Accumulation of CO2 in the respiratory gas or in the inner space of the patient interface 1 is thereby prevented, and the patient/user can inhale fresh respiratory gas.

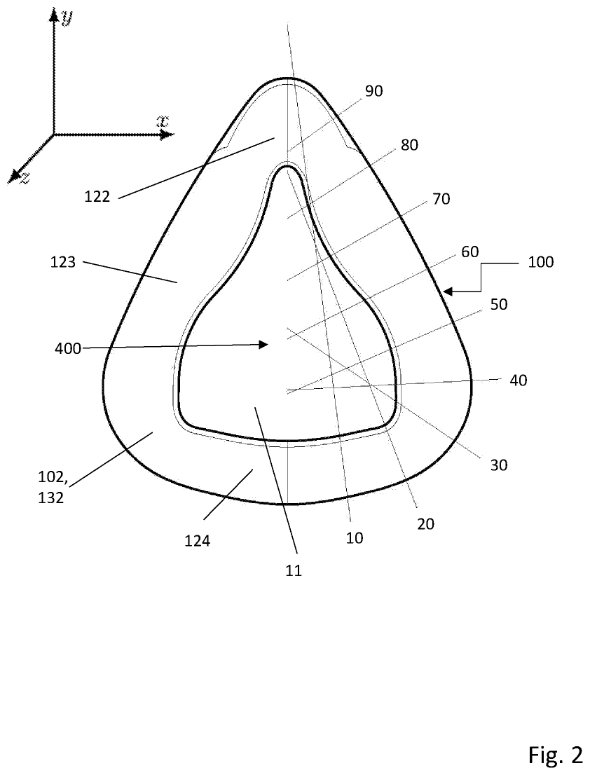

FIG. 2 shows a schematic representation of the sealing device 100 with different sectional planes 10, 20, 30 . . . 90. The sealing device 100 is shown in a plan view of the contact region 132, which is designed to bear on the face of a patient (not shown), and it is formed principally by the sealing lip 102. The sealing device has a vertical extent in a y direction and a horizontal extent in an x direction. The maximum vertical extent is greater than the maximum horizontal extent. The maximum vertical extent is 5-15 cm, and the maximum horizontal extent is 4-14 cm. The contact region 132 with the sealing lip 102 delimits (internally) the respiratory gas space 400 into which the mouth and/or nose of a patient (not shown) are inserted. The respiratory gas space 400 is formed by the interplay of mask body 2 and sealing device 100.

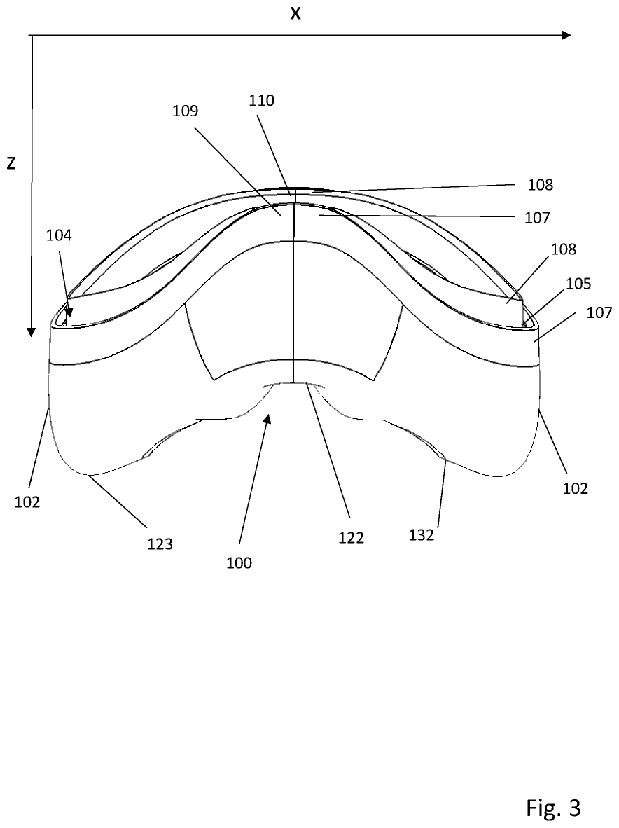

FIG. 3 shows a schematic representation of the sealing device 100 in a perspective view. The sealing device 100 is shown in a plan view from above. In an x direction, the maximum horizontal extent can be seen, which is substantially limited by the outer edge of the sealing lip 102.

In a z direction, the maximum depth can be seen, which is limited in the first instance by the contact region 132 of the sealing lip 102, which is designed to bear on the face of a patient (not shown). A further limit is provided by the walls 107 and 108 of the edge profile 104, which between them form a groove 105 and are designed for connection to a mask body (not shown).

The sealing device 100 has a vertical extent in a z direction and a horizontal extent in an x direction. The maximum vertical extent is smaller than the maximum horizontal extent. The maximum vertical extent is 5-15 cm, and the maximum horizontal extent is 4-14 cm.

The sealing device 100 has a height profile. This height profile can be seen, for example, from the profile of the walls 107 and 108 of the edge profile 104. The walls 107, 108, in the region of the tip 109 and the base 110 of the sealing device 100, are arranged near the x axis. In the region of the outer edge of the sealing lip 102, the walls 107, 108 are arranged away from the x axis.

The contact region 132 has a corresponding profile. The contact region 132 designed to bear on a nose bridge (not shown), the nose bridge region 122, is arranged near the x axis. The contact region 132 designed to bear on a chin (not shown) or upper or lower lip, the chin region 124, is arranged near the x axis.

The contact region 132 designed to bear on the cheeks (not shown) or the nasolabial fold, the nasolabial region 123, is arranged away from the x axis.

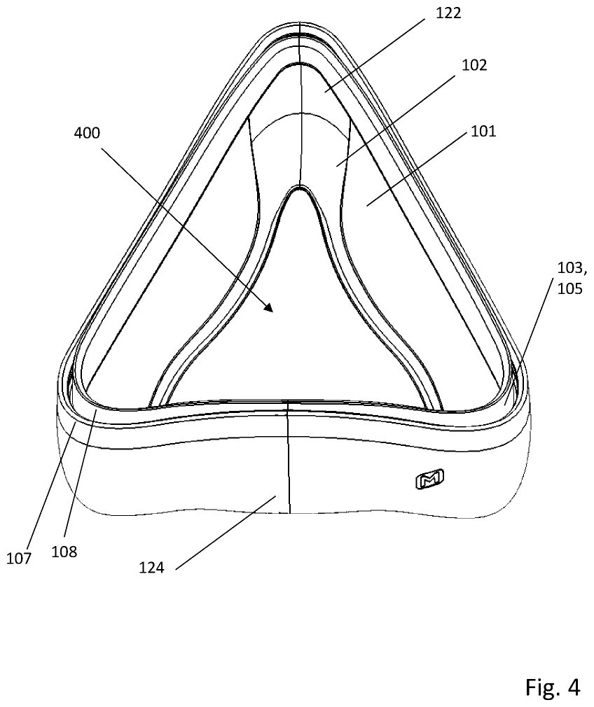

FIG. 4 shows a schematic representation of the sealing device 100 in a perspective view of the edge profile 103 as a contact point to the mask body 2. The sealing device 100 is shown in a plan view from below.

The approximately triangular basic shape of the sealing device 100 can be seen in this view. The sealing device 100 has an approximately U-shaped edge profile 103, which forms a fastening region for connecting the sealing device 100 to a mask body 2 by form-fit engagement. The edge profile 103 is formed by a peripheral open groove 105. The edge profile 103 has two profile walls 108, 107, which form the groove 105. The sealing membranes 101, 102 start out from the edge profile and point, in a direction way from the groove 105, toward a patient's face (not shown). The support membrane 101 is shorter and is covered at least in part (here completely) by the sealing membrane 102. This ensures that the patient's face is usually only in contact with the sealing membrane 102. It is also evident that the support membrane 101 is not continuous. The nose bridge region 122 and the chin region 124 are cut out from the support membrane 101.

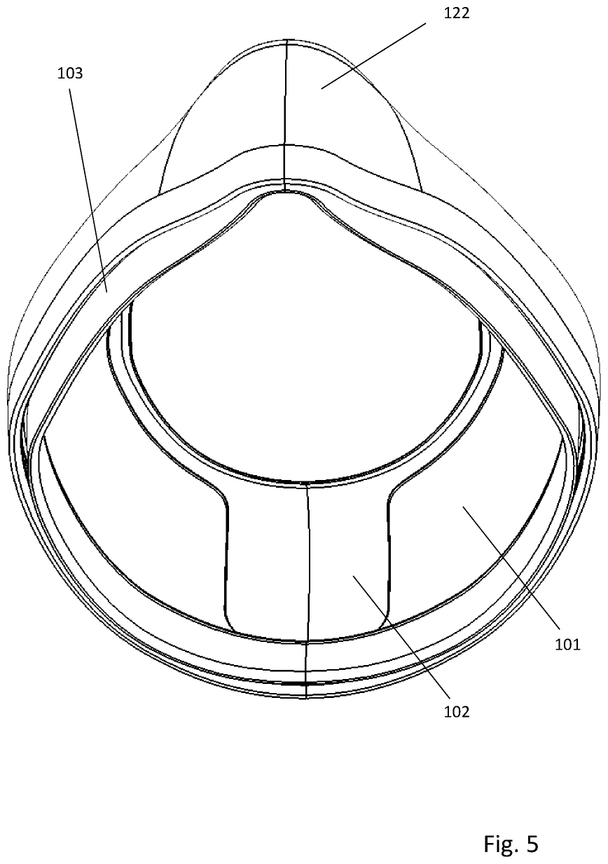

FIG. 5 shows a schematic representation of the sealing device 100 in a perspective view of the edge profile 103 as a contact point to the mask body 2. The sealing device 100 is shown in a plan view from above. It is evident that the support membrane 101 is not continuous. The chin region 124 is cut out from the support membrane 101. It will also be seen that the sealing membrane overlaps the support membrane.

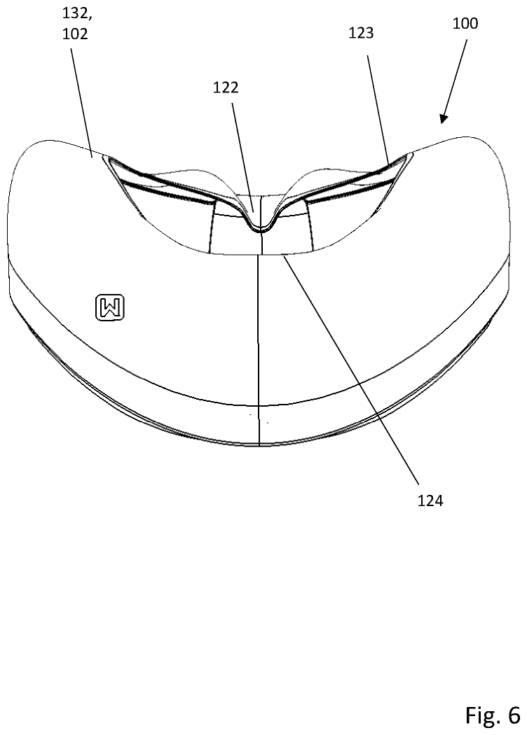

FIG. 6 shows a schematic representation of the sealing device 100 in a perspective view of the contact region 132 as a contact point to the patient. The sealing device 100 is shown in a plan view from below. The contact region 132 is formed in particular by the sealing membrane 102, which is formed in the contact region 132 as a sealing lip 112. The nose bridge region 122 and the chin region are cut out from the support membrane 101. Moreover, the height or the profile can be seen. The sealing device 100 has an indentation in the nose bridge region 122 and an indentation in the chin region 124. The lateral region, nasolabial region 123, is heightened by comparison. The region arranged on both sides immediately adjacent to the indentation in the nose bridge region 122 is likewise heightened.

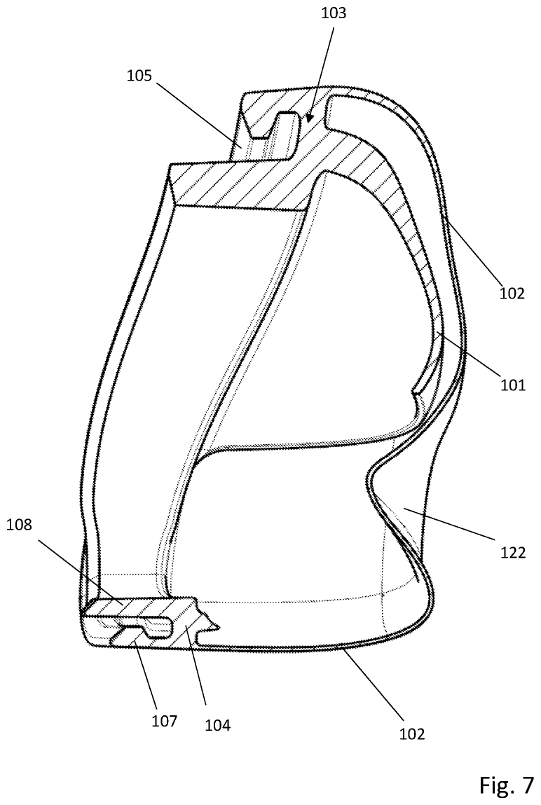

FIG. 7 shows a schematic representation of a section through a sealing device 100 according to FIG. 3 at the position 90, in the nose bridge region 122. The sealing device 100 has an approximately U-shaped edge profile 103, which forms a fastening region for connecting the sealing device 100 to a mask body 2 by form-fit engagement. The edge profile 103 is formed by a peripheral open groove 105. The edge profile 103 has two profile walls 108, 107, which form the groove 105.

It will be seen that the support membrane 101 is not continuous. The nose bridge region 122 is cut out from the support membrane 101. The sealing membrane 102 extends in a radiating fashion from the base 104 of the edge profile 103 and then bends round into a direction toward the inner space 400, the sealing membrane 102 at least in part forming a right angle to itself. The support membrane 101 likewise extends from the base 104 of the edge profile 103 substantially parallel to the sealing membrane 102. The support membrane 101 is thicker than the sealing membrane 102.

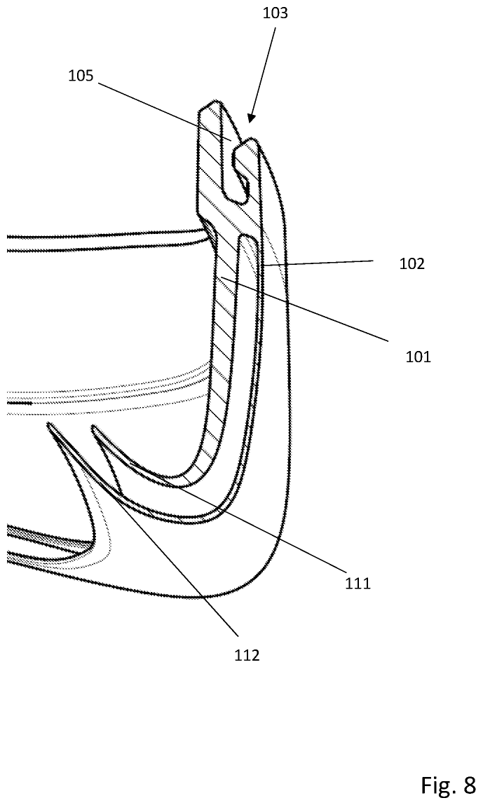

FIG. 8 shows a schematic representation of a section through a sealing device 100 according to FIG. 3 at the position 50, at the lower end of the nasolabial region 123. The sealing device 100 has an approximately U-shaped edge profile 103, which forms a fastening region for connecting the sealing device 100 to a mask body 2 by form-fit engagement. The edge profile 103 is formed by a peripheral open groove 105. It will be seen that the support membrane 101 is continuous here. The nasolabial region 123 is not cut out from the support membrane 101 and here supports the mask on the patient's face. The sealing membrane 102 extends in a radiating fashion from the base 104 of the edge profile 103 and then bends round into a direction toward the inner space 400, the sealing membrane 102 at least in part forming a right angle to itself. The support membrane 101 likewise extends from the base 104 of the edge profile 103 substantially parallel to the sealing membrane 102. The support membrane 101 is thicker than the sealing membrane 102, the support membrane 101 having a thickness profile. The support membrane 101 and the sealing membrane 102 end in sealing lips 111 and 112, respectively. The sealing lips can have the same thickness in a cross section.

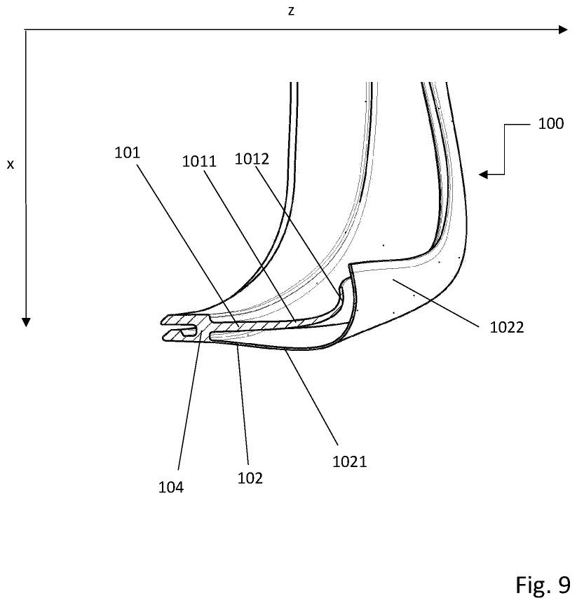

In the illustrative embodiment shown in FIG. 9, the sealing membranes 101, 102 are at least partially curved or rounded. In particular, the sealing membrane 101 and the sealing membrane 102 are designed radiating in a z direction from the base 104 and are then bent or rounded into an x direction. The sealing membrane 102 extends in a radiating fashion from the base 104 in a z direction and then bends round into an x direction, the sealing membrane 102 at least in part forming a right angle to itself. The sealing membrane 102 extends in a radiating fashion from the base 104 in a z direction with a first extent 1021 and then bends round into an x direction with a second extent 1022, the first extent 1021 being longer than the second extent 1022 of the sealing membrane 101, the first extent 1021 being more than 20 percent longer than the second extent 1022.

The support membrane 101 extends in a radiating fashion from the base 104 in a z direction and then bends round into an x direction, the support membrane 101 at least in part forming a right angle to itself. The support membrane 101 extends in a radiating fashion from the base 104 in a z direction with a first extent 1011 and then bends round into an x direction with a second extent 1012, the first extent 1011 being longer than the second extent 1012 of the sealing membrane 101, the first extent 1011 being more than 30 percent longer than the second extent 1012.

The sealing membrane 102 completely covers the support membrane 101. The second extent 1022 is therefore longer than the second extent 1012.

The surfaces of the sealing membranes 101, 102 can be structured or coated partially or completely in a manner that reduces friction. A friction-reducing design in the region of the sealing membranes 101, 102 can reduce a static and/or dynamic friction between the sealing membranes 101, 102.

A friction-reducing coating on the surface of the first support membrane 101 bearing on the inner sealing membrane 101 and on the second sealing membrane 102 and on the surface of the second sealing membrane 102 bearing on the outer and on the first support membrane 101 can in particular reduce the static friction between the sealing membranes 101, 102 and thereby reduce wear and promote the sealing function.

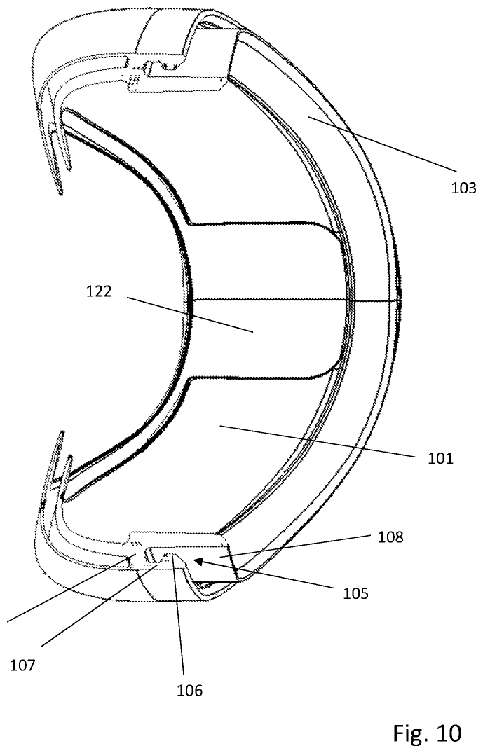

The sealing membranes 101, 102 shown in FIG. 10 originate in the base 104 and, spaced apart from each other, point downward and end in sealing lips 111, 112. The sealing membrane 102, substantially without offset, is designed as a continuation of the wall 107. However, the wall thickness of the sealing membrane 102 is smaller than the wall thickness of the wall 107. In the further course toward the sealing lip 112, the wall thickness of the sealing membrane 102 has a constant or only slightly decreasing wall thickness.

The sealing membrane 101, substantially with an offset, is designed as a continuation of the wall 106. The wall thickness of the support membrane 101 corresponds substantially to the wall thickness of the wall 108. In the further course toward the sealing lip 111, the wall thickness of the support membrane 101 has a decreasing wall thickness.

The distance between sealing membranes 102 and 101, over the range of the parallel extent, is substantially constant at about 2 mm.

The distance between the walls 107 and 108, over the range of the parallel extent, is substantially constant at about 2 mm.

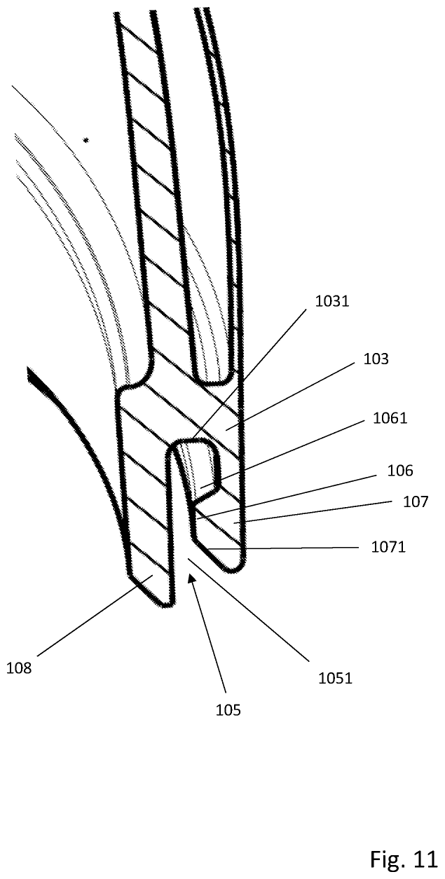

The sealing device 100 shown in FIG. 11 has an approximately U-shaped edge profile 103, which forms a fastening region for connecting the sealing device 100 to a mask body 2 by form-fit engagement. The edge profile 103 is formed by a peripheral open groove 105. The edge profile 103 has two profile walls 108, 107, which form the groove 105. The groove 105 has a funnel shape 1051. The groove 105 has a funnel shape 1051 by virtue of the fact that at least one of the profile walls 108, 107 has a beveled wall portion 1071 which, with its bevel (beveled wall portion) 1071, is set back from the opposite profile wall 108.

The profile wall 107 moreover forms a lug 206. The lug 206 is formed by a regional thickening of the cross section of the profile wall 107. The profile wall 107 has a beveled wall portion 1061 facing the base 1031 of the U-shaped edge profile 103. The beveled wall portion 1061, in its gradient relative to the wall 107, is steeper than the wall portion 1071. The beveled wall portion 1061 thus forms an undercut for the widening on the counter-profile 201 of the mask body 2. Between the lug and the base 1031, the edge profile has a receiving space 1032 for a corresponding thickening 203 of the counter-profile 201.

The edge profile 103 can receive a counter-profile 300 (not shown) of the mask body. The edge profile 103 and the counter-profile 200 of the mask body 2 thus produce a form-fit connection. On account of the elasticity of the edge profile 103, the connection can be produced and released repeatedly. To release the form-fit connection, in particular to withdraw the edge profile from the counter-profile 200 of the mask body, a tab or another means can be provided on the edge profile 103 or on the sealing device and can, for example, be gripped with two fingers in order to pull the edge profile 103 out of the counter-profile 200 of the mask body.

A lug 106 on a wall 107 of the edge profile 103 and a corresponding recess on the counter-profile 200 of the mask body establish a predetermined relative orientation between sealing device 100 and mask body 2. The wall 107 of the edge profile 103 having the lug 106 is shorter than the second wall 108. Both walls 107 and 108 originate from the base 104 of the edge profile 103 and, spaced apart from each other, point upward and thus between them form the groove 105.

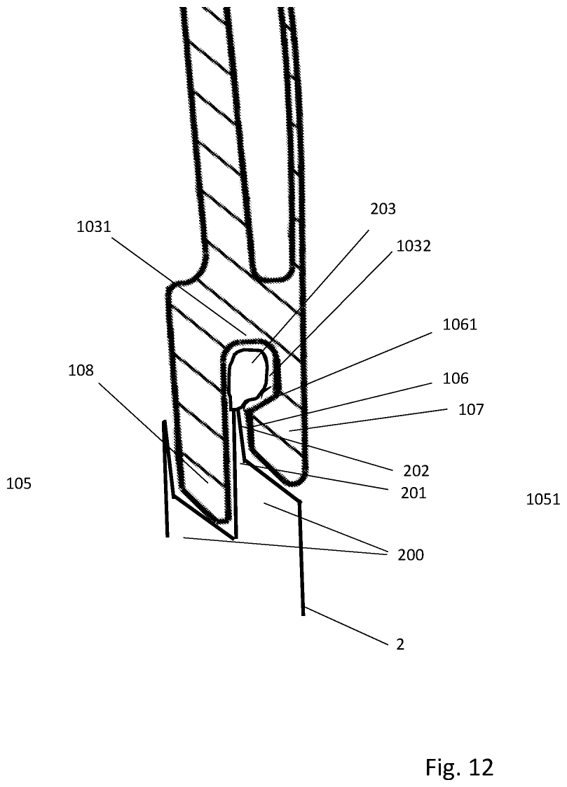

FIG. 12 shows the sealing device 100 with the U-shaped edge profile 103. FIG. 12 also shows here the form-fit connection of the sealing device 100 to a mask body 2. The mask body 2 for this purpose has a profile 200 which is designed such that it can be releasably connected to the sealing device 100 and produces a form-fit connection to the sealing device 100. In particular, the profile 200 has at least one counter-profile 201, which can be inserted into the groove 105. The counter-profile for this purpose has a web 202, on the end of which a thickening 203 is provided.

The thickening 203 of the counter-profile 201 is designed such that it can be inserted into the groove. The thickening 203 of the counter-profile 201 can be held in the groove by form-fit engagement or force-fit engagement or frictional engagement.

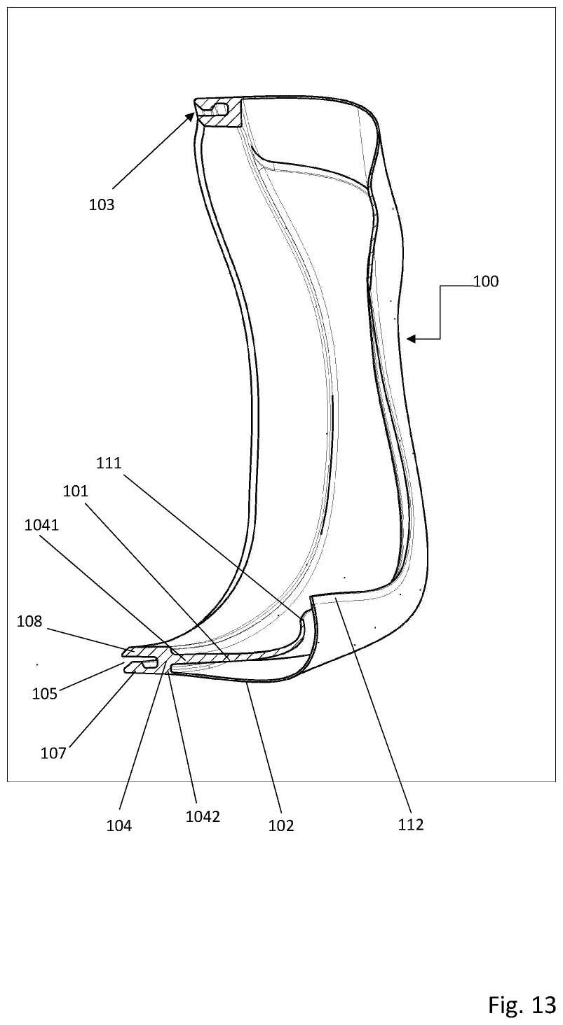

FIG. 13 shows a schematic representation of a section through a sealing device 100 according to FIG. 3 at the position 10. The section thus runs through the vertical of the sealing device 100 in the y direction.

Both walls 107 and 108 originate from the base 104 of the edge profile 103 and, spaced apart from each other, point in a z direction and thereby form between them the groove 105.

The sealing membranes 101,102 originate in the base 104 and, spaced apart from each other, point in another z direction (counter to 107, 108) and end in sealing lips 111, 112. The sealing membrane 102, substantially without an offset, is designed as a continuation of the wall 107. The sealing membranes 101, 102 form an elastic jacket-shaped wall portion.

However, the wall thickness of the sealing membrane 102 is smaller than the wall thickness of the wall 107. The wall thickness of the sealing membrane 102, in the further course toward the sealing lip 112, has a constant or only slightly decreasing wall thickness. The wall thickness of the sealing membrane 102 is in the range from about 0.2 mm to about 0.5 mm, preferably in the range from about 0.25 mm to about 0.45 mm, particularly preferably in the range from about 0.3 mm to about 0.4 mm.

The wall thickness of the support membrane 101, at the location 121 where the support membrane 101 originates from the base 104, is in the range from about 2.5 mm to about 1.4 mm, preferably in the range from about 2.1 to about 1.6 mm, particularly preferably in the range from about 2.0 mm to about 1.7 mm, very particularly preferably about 1.8 mm.

The wall thickness of the sealing lips 111, 112 is substantially identical.

The wall thickness of the support membrane 101, in the region of the sealing lip 111, is in the range from about 0.7 mm to about 0.35 mm, preferably in the range from about 0.65 mm to about 0.4 mm, particularly preferably in the range from about 0.6 mm to about 0.45 mm, very particularly preferably about 0.5 mm.

The distance between the walls 107 and 108, over the range of the parallel extent, is substantially constant at from about 1 mm to about 4 mm, preferably from about 1.4 mm to about 3 mm, particularly preferably from about 1.5 mm to about 1.8 mm.

The sealing membrane 101, substantially with an offset, is designed as a continuation of the wall 108. The wall thickness of the support membrane 101 corresponds substantially to the wall thickness of the wall 108. In the further course toward the sealing lip 111, the wall thickness of the support membrane 101 has a decreasing wall thickness.

The distance between sealing membranes 102 and 101 is about 2 mm at the base. From there, the sealing membranes 102 and 101 extend apart from each other with an angle 125 of greater than about 1°, preferably of from about 1.3° to about 4°, particularly preferably of about 3°.

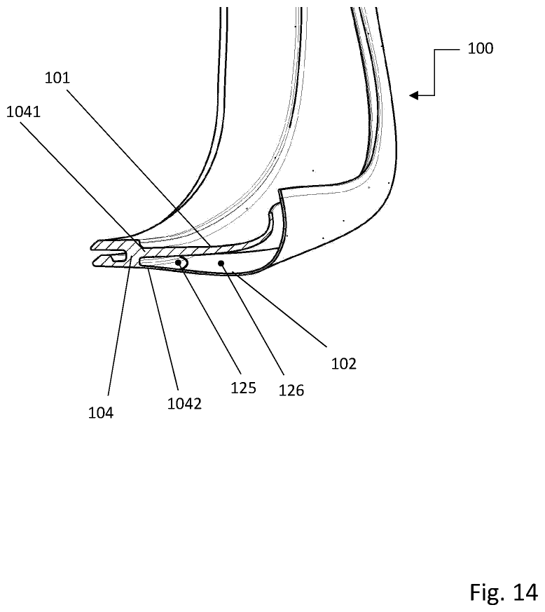

FIG. 14 shows a schematic representation of a further illustrative embodiment of a sealing device 100. The sealing device 100 is shown in FIG. 14 in a section along a plane which runs transverse to the longitudinal axis of the sealing device 100.

The sealing device 100 is produced from silicone or another elastic material. The sealing device 100 comprises a first support membrane 101 and a second sealing membrane 102.

The second sealing membrane 102 forms an elastic jacket-like wall portion which delimits the sealing device 100 to the outside and at least partially covers or overlaps the first support membrane 101. The elasticity of the elastic jacket-shaped wall portion permits a movement of the sealing membranes 101, 102 relative to the fastening region of the sealing device 100 formed by the edge profile 103, in particular a displacement perpendicular to the longitudinal axis and/or a tilting or pivoting about axes perpendicular to the longitudinal axis.

The distance between the sealing membranes 101 and 102, over the range of the common extent, is substantially constant at from about 1 mm to about 4 mm, preferably from about 1.4 mm to about 3 mm, particularly preferably from about 1.5 mm to about 1.8 mm, or about 2 mm. In the illustrative embodiment shown, the distance between the sealing membranes 101 and 102 at the origin 1041, 1052 is about 1.5 mm. From there, the sealing membranes 101 and 102 extend apart from each other with an angle 125 of greater than about 1°, preferably of from about 1.3° to about 4°, particularly preferably of about 3°. The distance between the sealing membranes 101 and 102 increases over the range of the common extent, as a result of the angle 125, and at least in part is at least from about 0.5 mm to about 5 mm, preferably from about 1.4 mm to about 3 mm, particularly preferably from about 1.5 mm to about 2.0 mm. The slight angle 125 has the effect that the sealing membranes 101 and 102 do not adhere to each other or run together but instead diverge. In this way, respiratory gas is able to flow through the interspace 126 at least partially surrounded by the sealing membranes 101, 102. The flow of respiratory gas through the interspace 126 at least partially surrounded by the sealing membranes 101, 102 has at least two advantageous effects. Firstly, the therapeutic pressure applied can blow the sealing membrane 102 at least slightly outward or press it against the patient's face. Secondly, an accumulation of condensing moisture from the respiratory air or the respiratory gas is at least lessened or partially prevented by the flow through the interspace 126.

A mobility, in particular a displaceability, of the sealing membranes 101, 102 and of the sealing lips 111, 112 relative to each other is provided by the elasticity of the elastic jacket-shaped wall portion of the sealing membranes 101, 102 and of the sealing lips 111, 112.

An orientation of the sealing device 100 on a face (not shown) is effected by an approximately dome-shaped curvature of the sealing lips 111, 112, the curvature being design circumferentially in the direction of the face opening 400 (FIG. 2). The dome-shaped curvature of the sealing membranes 101, 102 is also possible with a displaceability of the sealing device 100.

The following applies to all embodiments:

The support membrane 101 can have a material which is considerably stiffer or less elastic than the material or the materials of the sealing membrane 102. In this case, the material thickness of the membrane 101 can have a reduced thickness in one cross section.

The first support membrane 101 can be joined to the edge profile 103, in particular adhesively bonded, welded or otherwise cohesively bonded at a contact location. Alternatively, the support membrane 101 and the edge profile 103 can be cohesively joined to each other during an injection molding process or another casting process.

Alternatively, the support membrane 101 and the edge profile 103 are produced from the same material and in particular at the same time and integrally joined from the outset. The second sealing membrane 102 and the edge profile 103 can also be produced from the same material and in particular at the same time and integrally joined from the outset.

To sum up, the present invention provides:
1. A patient interface with a sealing device, wherein the sealing device comprises a support membrane and a sealing membrane, the sealing membrane being designed at least in part to bear on a skin of a patient and the support membrane extending at least in part adjacent to the sealing membrane, and a distance between the support membrane and the sealing membrane being at least in part such that an interspace is formed.
2. The patient interface of item 1, wherein the sealing device has an edge profile with a base, the sealing membrane and the support membrane extending, spaced apart from each other, starting from the common base.
3. The patient interface of item 1 or item 2, wherein the sealing has an approximately U-shaped edge profile, which forms a fastening region for connecting the sealing device by form-fit engagement to a mask body.
4. The patient interface of item 3, wherein the fastening region is formed by a peripheral open groove.
5. The patient interface of any one of items 3 and 4, wherein the edge profile has two profile walls which form a funnel-shaped groove.
6. The patient interface of item 5, wherein the groove has a funnel shape by virtue of the fact that at least one of the profile walls has a beveled wall portion which, with its bevel (beveled wall portion), is set back from an opposite profile wall, and the profile wall has a lug.
7. The patient interface of item 6, wherein the profile wall has a beveled wall portion which faces the base of the U-shaped edge profile, the beveled wall portion has an undercut for a widening on a counter-profile of a mask body, and the edge profile can thus receive a counter-profile of a mask body.
8. The patient interface of at least one of the preceding items, wherein the membranes start from the edge profile and point in a direction away from the groove, a sealing membrane originates from the edge profile all about the periphery, and in some regions there is no support membrane originating from the edge profile, and therefore the support membrane is not continuous.
9. The patient interface of at least one of the preceding items, wherein the support membrane is shorter than the sealing membrane, and the support membrane is at least in part covered by the sealing membrane.
10. The patient interface of least one of the preceding items, wherein a contact region to a patient's skin is formed by the sealing membrane, which is formed as a sealing lip in the contact region.
11. The patient interface of at least one of the preceding items, wherein the sealing membrane extends in a radiating fashion, starting from a base of an edge profile, and then bends round in one direction, the sealing membrane at least in part forming a right angle to itself.
12. The patient interface of at least one of the preceding items, wherein the membranes spaced apart from each other, originate in a base, the sealing membrane is designed as a continuation of a wall, the wall thickness of the sealing membrane is smaller than the wall thickness of the wall, and the support membrane is at least in part of thicker cross section than the sealing membrane.
13. The patient interface of at least one of the preceding items, wherein the wall thickness of the sealing membrane, in its further course, has a constant or only slightly decreasing wall thickness.
14. The patient interface of at least one of the preceding items, wherein the support membrane, substantially with an offset, is designed as a continuation of a wall, the wall thickness of the support membrane substantially corresponds to the wall thickness of the wall, and the wall thickness of the support membrane in its further course (toward the sealing lip) has a decreasing wall thickness.
15. The patient interface of at least one of the preceding items, wherein the wall thickness of the sealing membrane, in a course toward a sealing lip, has a constant or only slightly decreasing wall thickness, a wall thickness of the sealing membrane being in the range of from 0.15 mm to 0.6 mm.
16. The patient interface of at least one of the preceding items, wherein the wall thickness of the support membrane is in a range of from 2.9 mm to 1.4 mm at a location where the support membrane originates from the base.
17. The patient interface of at least one of the preceding items, wherein the wall thickness of the support membrane is in a range of from 2.1 mm to 1.6 mm, particularly preferably in a range of from 2.0 mm to 1.7 mm, very particularly preferably 1.8 mm, and the wall thickness of the support membrane in the region of the sealing lip is in the range from 0.7 mm to 0.25 mm.
18. The patient interface of at least one of the preceding items, wherein the wall thickness of the sealing lips is substantially identical.
19. The patient interface of at least one of the preceding items, wherein the distance between the sealing membrane and the support membrane at the base is from 0.5 mm to 3.5 mm, and from there the membranes extend apart from each other with an angle of greater than 1°, preferably of from 1.3° to 4°, particularly preferably of 3°.
20. The patient interface of at least one of the preceding items, wherein, over a region of a common extent, the distance between the membranes is substantially from 1 mm to 4 mm, preferably from 1.4 mm to 3 mm, particularly preferably from 1.5 mm to 1.8 mm or 2 mm.
21. The patient interface of at least one of the preceding items, wherein the sealing device is produced from silicone or another elastic material, the sealing membrane forms an elastic jacket-like wall portion which delimits the sealing device to the outside and at least partially covers or overlaps the support membrane, and the elasticity of the elastic jacket-like wall portion permits a movement of the membranes relative to a region of the sealing device formed by an edge profile, in particular a displacement perpendicular to the longitudinal axis and/or a tilting or pivoting about axes perpendicular to the longitudinal axis.
22. The patient interface of at least one of the preceding items, wherein a mask body and the sealing device together form an inner space or respiratory gas space into which a face of a user/patient at least partially protrudes during use of the mask on the face, the inner space of the patient interface is connected to an outer region of the patient interface in a gas-conducting manner via an exhalation system and, by way of a hose connector, the patient interface is connected to a ventilator in a gas-conducting manner via a breathing hose, the sealing device comprising a contact region which is designed to bear sealingly on a patient's face and is formed principally by a sealing lip.
23. The patient interface of claim item 22, wherein the contact region has a profile which is designed such that the contact region is designed to bear on a nose bridge and to bear along a nasolabial fold and along an upper lip or lower lip or a chin region of a patient.

What is claimed is:

1. A patient interface with a sealing device, wherein the sealing device comprises a support membrane and a sealing membrane, the sealing membrane being designed at least in part to bear on a skin of a patient and the support membrane extending at least in part adjacent to the sealing membrane, and a distance between the support membrane and the sealing membrane being at least in part such that an interspace is formed.

2. The patient interface of claim 1, wherein the sealing device has an edge profile with a base, the sealing membrane and the support membrane extending, spaced apart from each other, starting from a common base.

3. The patient interface of claim 1, wherein the sealing device has an approximately U-shaped edge profile, which forms a fastening region for connecting the sealing device by form-fit engagement to a mask body.

4. The patient interface of claim 3, wherein the fastening region is formed by a peripheral open groove.

5. The patient interface of claim 2, wherein the edge profile has two profile walls which form a funnel-shaped groove.

6. The patient interface of claim 5, wherein the groove has a funnel shape by virtue of the fact that at least one of the profile walls has a beveled wall portion which, with its bevel (beveled wall portion), is set back from an opposite profile wall, and the profile wall has a lug.

7. The patient interface of claim 6, wherein the profile wall has a beveled wall portion which faces the base of the U-shaped edge profile, the beveled wall portion has an undercut for a widening on a counter-profile of a mask body, and the edge profile can thus receive a counter-profile of a mask body.

8. The patient interface of claim 1, wherein the membranes start from an edge profile and point in a direction away from a groove, the sealing membrane originates from the edge profile all about a periphery, and in some regions there is no support membrane originating from the edge profile, and therefore the support membrane is not continuous.

9. The patient interface of claim 1, wherein the support membrane is shorter than the sealing membrane, and the support membrane is at least in part covered by the sealing membrane.

10. The patient interface of claim 1, wherein a contact region to a patient's skin is formed by the sealing membrane, which is formed as a sealing lip in the contact region.

11. The patient interface of claim 1, wherein the sealing membrane extends in a radiating fashion, starting from a base of an edge profile, and then bends round in one direction, the sealing membrane at least in part forming a right angle to itself.

12. The patient interface of claim 1, wherein the membranes spaced apart from each other, originate in a base, the sealing membrane is designed as a continuation of a wall, a wall thickness of the sealing membrane is smaller than a wall thickness of the wall, and the support membrane is at least in part of thicker cross section than the sealing membrane.

13. The patient interface of claim 12, wherein the wall thickness of the sealing membrane, in its further course, has a constant or only slightly decreasing wall thickness.

14. The patient interface of claim 1, wherein the support membrane, substantially with an offset, is designed as a continuation of a wall, a wall thickness of the support membrane substantially corresponds to a wall thickness of the wall, and a wall thickness of the support membrane in its further course (toward a sealing lip) has a decreasing wall thickness.

15. The patient interface of claim 1, wherein a wall thickness of the sealing membrane, in a course toward a sealing lip, has a constant or only slightly decreasing wall thickness, the wall thickness of the sealing membrane being in the range from 0.15 mm to 0.6 mm.

16. The patient interface of claim 1, wherein a wall thickness of the support membrane is in a range of from 2.9 mm to 1.4 mm at a location where the support membrane originates from a base.

17. The patient interface of claim 1, wherein a wall thickness of the support membrane is in a range of from 2.1 mm to 1.6 mm, and a wall thickness of the support membrane in a region of a sealing lip is in the range from 0.7 mm to 0.25 mm.

18. The patient interface of claim 1, wherein a wall thickness of sealing lips is substantially identical.

19. The patient interface of claim 1, wherein a distance between the sealing membrane and the support membrane at a base is from 0.5 mm to 3.5 mm, and from there the membranes extend apart from each other with an angle of greater than 1°.

20. The patient interface of claim 1, wherein, over a region of a common extent, a distance between the membranes is substantially from 1 mm to 4 mm.
